(19) United States
(12) Patent Application Publication
(10) Pub. No.: US 2022/0192979 A1
(43) Pub. Date: Jun. 23, 2022

(54) Liquid Micro-Dosing Product Containing an Effective Amount of Cannabis and Process for Making the Same

(71) Applicant: Liquid Secrets, LLC, Oklahoma City, OK (US)

(72) Inventor: Michael Keith Gaya, Oklahoma City, OK (US)

(21) Appl. No.: 17/129,519

(22) Filed: Dec. 21, 2020

(51) Int. Cl. A61K 9/00; A61K 31/05; A61K 47/44; A61K 31/352

(52) U.S. Cl. CPC A61K 9/0095; A61K 31/352; A61K 47/44; A61K 31/05

(57) ABSTRACT

A liquid micro-dosing product including a distillate comprising an effective amount of a cannabinoid, water, polysorbate 80, lecithin, vegetable oil and flavor powder is disclosed. A method of producing a liquid micro-dosing product including the following steps: mixing water and polysorbate 80 to form an aqueous solution; mixing vegetable oil, lecithin and a distillate comprising an effective amount of cannabinoid to form a distillate base solution; homogenizing the aqueous solution and the distillate base solution together to form a homogenized solution; and mixing flavor powder into the homogenized solution is disclosed. A method of treating a medical condition in a subject in need of such therapy is also disclosed. The method including the step of administering to the subject a beverage containing a liquid micro-dosing product comprising a distillate comprising an effective amount of a cannabinoid; water; polysorbate 80; lecithin; vegetable oil; and flavor powder.

### BACKGROUND

The Cannabis plant is a genus of flowering plants. There are a variety of different species within the genus, including, but not limited to, Cannabis sativa, Cannabis indica and Cannabis ruderalis for example. Cannabinoids are chemical compounds found in Cannabis plants that interact with cannabinoid receptors in the brain and body to create various effects. One such cannabinoid is Δ⁹-tetrahydrocannabinol (“THC”), which has been isolated, synthesized and studied due to its psychoactive effects. Other cannabinoids which have been isolated, synthesized and studied include Δ⁸-THC, cannabinol (“CBN”) and cannabidiol (“CBD”) for example. CBN is a mildly psychoactive cannabinoid while CBD is not known to produce any psychoactive effect. The chemical formula for Δ⁹-THC, Δ⁸-THC, CBN and CBD are shown below and in FIGS. 1-4:

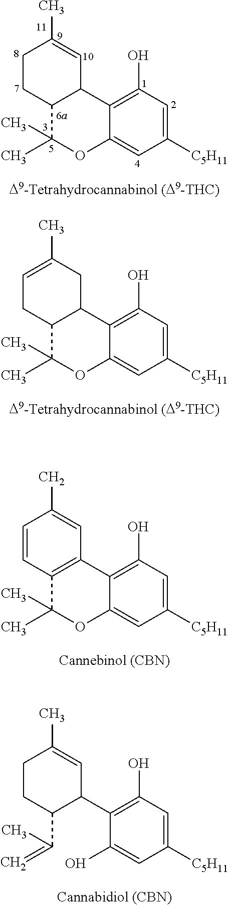

It is known that cannabinoids, including THC and CBD may have various health and medical benefits. The health and medical benefits of CBD generally include, reducing seizures in people with epilepsy, reducing insomnia, pain relief, reducing inflammation, reducing chronic pain and anxiety relief, among various other medical benefits. The health and medical benefits of THC generally include, reducing seizures in people with epilepsy, reducing insomnia, pain relief, reducing inflammation, reducing chronic pain, reducing anxiety, relief, elation, relaxation, sedation, pain relief, energy, hunger, drowsiness, slowed perception of time and laughter, among various other benefits. There are a variety of medical conditions for which CBD and THC may offer health and medical benefits. These medical conditions include, but are not limited to, post traumatic stress disorder, neuropathic and chronic pain, insomnia, nausea, inflammation, arthritis, migraines, cancer, crohn's disease, fibromyalgia, alzheimer's disease, multiple sclerosis, glaucoma, attention deficit hyperactivity disorder (“ADHD”), sleep apnea and appetitie loss for example.

Cannabis is used recreationally and medicinally and may be administered to a person in a variety of different ways. For example, Cannabis may be administered to a person by smoking, vaporizing, eating, drinking or inhaling Cannabis or products that contain Cannabis including extracts and distillates that contain cannabinoids such as THC, CBD or CBN for example. Unfortunately, however, traditional methods and traditional products for orally administering Cannabis via food and beverage products are not effective, efficient or reliable at delivering accurate micro-doses of cannabinoids such as THC, CBD or CBN to a person. This is because the cannabinoids that are mixed within food and beverage products have significantly reduced bioavailability (often less than 50% bioavailability and typically no greater than 75% bioavailability). Bioavailability is the proportion of the cannabinoid that enters the blood stream of a person and is able to have an active effect). The bioavailability of such cannabinoids when mixed with food and beverage products and consumed orally by a person is reduced because of low absorption rates into the blood stream and high metabolism rates of the food and beverage products in the digestive system. Moreover, the cannabinoids that are mixed into the food or beverage products are often not evenly or sufficiently mixed or distributed throughout the food or beverage product, resulting in gritty and bad tasting foods and beverages, settling of the cannabinoids in the beverage or unequal distribution of the cannabinoids in the food product resulting in uneven and inconsistent delivery of the cannabinoid to the person.

As a result, such traditional methods and products for orally administering Cannabis via food and beverage products (and processes for making such Cannabis infused food and beverage products) do not provide a safe, reliable or effective way for a person to accurately self administer a specific and accurate dosage of a cannabinoid, in particular a specific and accurate micro-dosage of cannabinoid such as THC or CBD for example. For people that desire only a relatively small amount of cannabinoid such as THC or CBD for medical or recreational purposes traditional Cannabis infused food or beverage products are not safe, reliable or efficient. Thus, decreasing the usefulness and safety of such traditional food and beverage products containing Cannabis.

To that end, it would be advantageous to provide an improved liquid micro-dosing product that contains an effective amount of a cannabinoid and an improved process for making the same. The improved liquid micro-dosing product described and claimed herein is configured to be administered orally by adding the improved liquid micro-dosing product directly into a beverage of choice for consumption by a person for both medical and recreational purposes. The improved liquid micro-dosing product has improved cannabinoid bioavailability (with little to no separation or settling of the cannabinoid in the liquid micro-dosing product or beverage) for efficient and accurate delivery of micro-doses of a cannabinoid into the blood stream of a person and as a result of the improved homogenization process used to produce the improved liquid micro-dosing product described and claimed herein. Thereby providing significant improvements over known Cannabis infused liquid beverage products and processes for both medical and recreational purposes.

### BRIEF DESCRIPTION OF THE DRAWINGS

Several embodiments of the present disclosure are hereby illustrated in the appended drawings. It is to be noted however, that the appended drawings only illustrate certain embodiments and are therefore not intended to be considered limiting of the scope of the present disclosure.

FIG. 1 depicts the chemical compound for Δ⁹-tetrahydrocannabinol (Δ⁹-THC).

FIG. 2 depicts the chemical compound for Δ⁸-tetrahydrocannabinol (Δ⁸-THC).

FIG. 3 depicts the chemical compound for cannabinol (CBN).

FIG. 4 depicts the chemical compound for cannabidiol (CBD).

FIG. 5 and FIGS. 6A-6O depict tables showing formulas for a variety of different batch sizes of liquid micro-dosing product produced in accordance with the inventive concepts disclosed and claimed herein. The tables depicts numbers in gram weight.

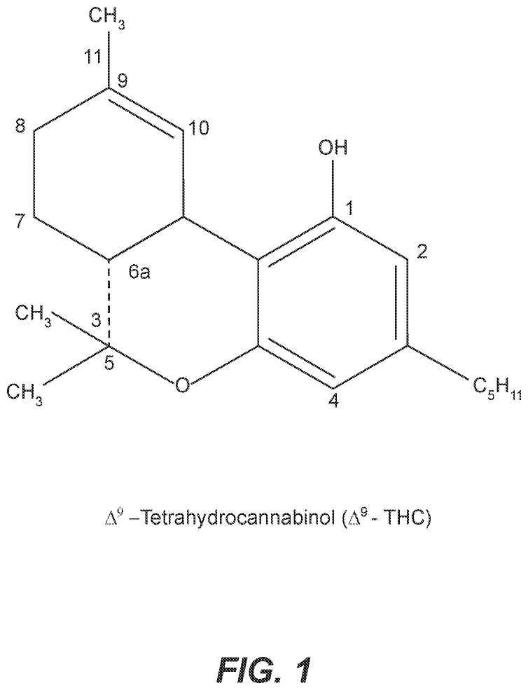

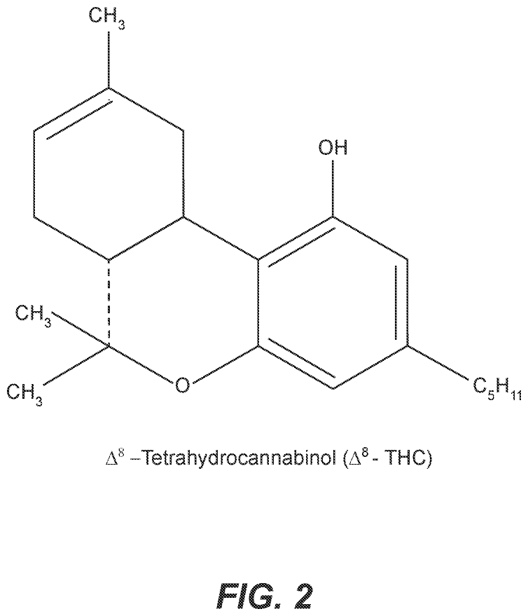

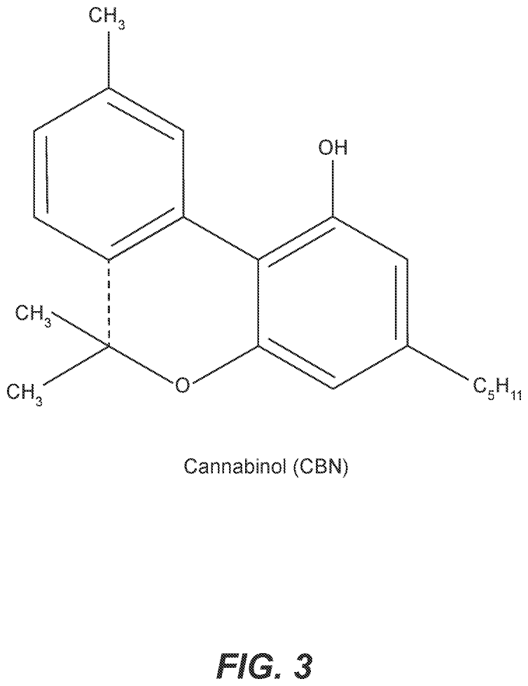

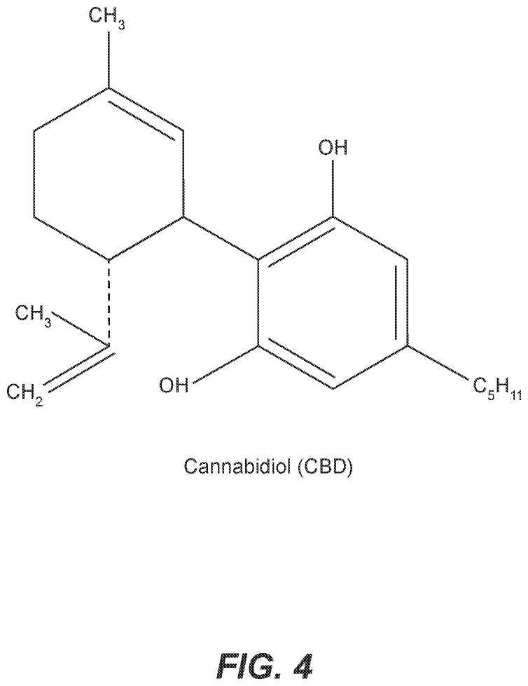

### DETAILED DESCRIPTION

The present disclosure describes methods and compositions comprising an effective amount of a cannabinoid for recreational use and for use in the treatment of medical conditions including, but not limited to, post traumatic stress disorder, neuropathic and chronic pain, insomnia, nausea, inflammation, arthritis, migraines, cancer, crohn's disease, fibromyalgia, alzheimer's disease, multiple sclerosis, glaucoma, attention deficit hyperactivity disorder (“ADHD”), sleep apnea and appetitie loss, as well as for reducing seizures in people with epilepsy, reducing insomnia, pain relief, reducing inflammation, reducing chronic pain, reducing anxiety, increasing elation, increasing relaxation, sedation, pain relief, energy, hunger, drowsiness, slowed perception of time and laughter for example.

Before further describing various embodiments of the compounds, compositions and methods of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure.

All of the compounds, compositions, and methods of application and use thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions and/or methods and in the steps or in the sequence of steps of the methods described herein without departing from the concept, spirit, and scope of the inventive concepts.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1,3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The term “pharmaceutically acceptable” refers to compounds and compositions which are suitable for administration to humans without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, and diluents which may improve solubility, deliverability, dispersion, stability, and/or conformational integrity of the compounds or conjugates thereof. As used herein a “pharmaceutically acceptable carrier” is a pharmaceutically acceptable solvent, suspending agent, vehicle, or diluent for delivering the cannabinoid of the present disclosure to a subject. The carrier may be liquid or solid and is selected with the planned manner of administration in mind. Examples of pharmaceutically acceptable carriers that may be utilized with the cannabinoid disclosed herein include but are not limited to polyethylene glycol (PEG) of various molecular weights, liposomes, ethanol, dimethyl sulfoxid (DMSO), aqueous buffers, oils, and combinations thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. In addition, the compositions of the present disclosure may be designed to provide delayed, controlled, extended, and/or sustained release using formulation techniques which are well known in the art.

The terms “therapeutic composition” and “pharmaceutical composition” refer to an cannabinoid-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of a cannabinoid of the present disclosure which is sufficient to exhibit a desired recreational effect or a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and purpose of the use and the severity of any condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

In certain embodiments the improved liquid micro-dosing product is configured to be administered orally by adding the improved liquid micro-dosing product directly into a beverage of choice for consumption by a person for both medical and recreational purposes. Alternatively, the improved liquid micro-dosing product may be formulated as part of a beverage instead of being added to a variety of different beverages, for example. The improved liquid micro-dosing product has improved cannabinoid bioavailability (with little to no separation or settling of the cannabinoid in the liquid micro-dosing product or beverage) for efficient and accurate delivery of micro-doses of a cannabinoid into the blood stream of a person and as a result of the improved homogenization process used to produce the improved liquid micro-dosing product described and claimed herein. Thereby providing significant improvements over known Cannabis infused liquid beverage products and processes for both medical and recreational purposes.

In certain embodiments, the liquid micro-dosing product includes a distillate having an effective amount of a cannabinoid; water; polysorbate 80; lecithin; vegetable oil (coconut oil, olive oil, MCT oil or any food grade high fat oil); and flavor powder and/or flavor liquid. The liquid micro-dosing product may be produced as a stand-alone product configured to be added to any beverage, including but not limited to juice, soda, coffee, tea, energy drinks, water and alcoholic beveragages for example. Alternatively, some embodiments are of a beverage containing a liquid micro-dosing product comprises a distillate comprising an effective amount of a cannabinoid; water; polysorbate 80; lecithin; vegetable oil (coconut oil, olive oil, MCT oil or any food grade high fat oil); and flavor powder and/or flavor liquid. In some embodiments, each of the water; polysorbate 80; lecithin; vegetable oil (coconut oil, olive oil, MCT oil or any food grade high fat oil); and flavor powder and/or flavor liquid are present in the liquid micro-dosing product at about 0.260% to 0.280%. In some embodiments, the percentage of polysorbate 80, lecithin, vegetable oil and flavor powder and/or flavor liquid may be in a range of about 0.346% to 0.530% or in such greater or lesser range depending upon the amount of liquid micro-dosing product to be produced in accordance with the inventive concepts disclosed herein. Likewise, in some embodiments, the distillate comprising an effective amount of cannabinoid is present in the liquid micro-dosing product at about 0.05% to 0.20%. In some embodiments, the percentage of distillate comprising an effective amount of cannabinoid may also be present in a range of about 0.0641% to 0.015% or in such greater or lesser range depending upon the amount of liquid micro-dosing product to be produced in accordance with the inventive concepts disclosed herein.

In certain embodiments, the distillate containing an effective amount of cannabinoid includes an effective amount of Δ⁹-tetrahydrocannabinol, while in other embodiment the cannabinoid is Δ⁸-tetrahydrocannabinol, cannabinol or cannabidiol for example. It should be understood by one of skill in the art that the inventive concepts disclosed herein are not limited by the type of cannabinoid and that any cannabinoid or a variety or combination of cannabinoids may be used with the liquid micro-dosing product and process disclosed and claimed herein.

Distillate is a Cannabis extract in which the final product has been substantially purified of all materials and compounds except for specific cannabinoids. For example, distillate can be formed to contain pure or substantially pure Δ⁹-tetrahydrocannabinol, Δ⁸-tetrahydrocannabinol, cannabinol or cannabidiol. Distillates are Cannabis extracts that have been winterized, decarboxylated and then distilled or purified. Distillate is the base ingredient of most edibles and typically lacks any terpenes which provide a flavor, taste or aroma. One benefit of having the natural terpenes removed is being able to have complete control over the final product's taste and smell. It is a potent Cannabis oil that can be used by itself or with other Cannabis goods and products.

The process of making a Cannabis distillate generally starts with crude oil extraction, which is any process where the cannabinoids are separated from the Cannabis plant material. A crude extraction typically involves either a physical means of separation or a chemical means of separation. Physical separation techniques, such as sieving or rosin, tend to yield concentrates containing more plant impurities than chemical-based extraction methods, namely Butane Hash Oil (BHO) or supercritical fluid carbon dioxide extraction. Whether the cannabinoids are separated by physical or chemical means, the crude extract produced contains impurities that must be removed before the oil can be separated into its individual cannabinoids. The next major step in producing distillate is called winterization. It is a method to purify the crude extract of byproducts: plant waxes, fats, lipids, and chlorophyll. The crude extract is mixed with ethanol. The solution is then placed in a very cold environment for 24 to 48 hours. The impurities coagulate in the cold temperature and precipitate, or separate, falling to the bottom of the container. The crude extract and ethanol solution is then passed through a filter. After filtering, the ethanol is removed. Ethanol can be removed using a variety of techniques, such as a rotary evaporator or a falling film evaporator. The extract at this point wouldn't be very potent. THC, for example, is the well-known compound and active cannabinoid that produces an intoxicating effect. However, it's tetrahydrocannabinolic acid (THCA), that's found at this stage. THCA doesn't produce an intoxicating effect. THCA becomes THC after heat is applied. This process is called decarboxylation. THCA isn't the only cannabinoid that needs to be decarboxylated in order to interact effectively with the human body. All cannabinoids in their acid form must first be decarboxylated.

Decarboxylation is the process of removing the carboxylic acid from a cannabinoid's chemical compound. A cannabinoid is decarboxylated when it's heated to the point of eliminating the carboxylic acid. By removing that acid group, the cannabinoid can readily interact within the body and bind to the receptors in the nervous system, specifically, the cannabinoid type 1 (CB1) and cannabinoid type 2 (CB2) receptors. The point of decarboxylation depends primarily on time and temperature. For example, THCA begins to decarboxylate into THC when it's exposed to heat at 220 degrees Fahrenheit, or 104.44 degrees Celsius, or to an open flame. The final steps of making a distillate are the actual Cannabis distillation process. Using vacuum pressure and heat, individual cannabinoids and terpenes can be separated from the decarboxylated extract according to their unique boiling points and molecular weights. In a vacuum environment, where the pressure can be strictly controlled, the boiling point of can be achieved at much lower temperatures to help prevent the loss of potency.

Polysorbate 80 is a nonionic surfactant and emulsifier used to stabilize aqueous formulations. The synthetic compound is a viscous, water soluble liquid. Polysorbate 80 is derived from polyethoylated sorbitan and oleic acid. The hydrophilic groups in this compound are polyethers known as polyoxyethylene groups, which are polymers of ethylene oxide. The numeric designation following polysorbate refers to the lipophilic group, in this case, the oleic acid. The full chemical names for polysorbate 80 are: polyoxyethylene (20) sorbitan monooleate (x)-sorbitan mono-9-octadecenoate poly(oxy-1,2-ethanediyl).

Lecithin is a generic term used to designate fatty compounds occurring in animal and plant tissue which are amphiphilic, meaning that the substance attracts both water and fatty substances (and so are both hydrophilic and lipophilic). Lecithin is used for smoothing food textures, emulsifying and homogenizing liquid mixtures and repelling sticking materials. Lecithins are mixtures of glycerophospholipids including phosphatidylcholine, phosphatidylethanolamine, phosphatidylinositol, phosphatidylserine, and phosphatidic acid. Commercial lecithin, as used by food manufacturers, is a mixture of phospholipids in oil. The lecithin can be obtained by water degumming the extracted oil of seeds. It is a mixture of various phospholipids, and the composition depends on the origin of the lecithin. A major source of lecithin is soybean or sunflower oil.

Vegetable oils high in medium chain triglycerides (MCTs), including medium chain triglyceride (MCT) oil and coconut oil are edible oils high in MCTs that have been extracted from a vegetable. Triglycerides are named after their chemical structure, specifically the length of their fatty acid chains. Triglycerides consist of a glycerol molecule and there fatty acids. The majority of fat in a diet is made up of long-cain fatty acids, which contain 13-21 carbons. Short chain fatty acids typically contain fewer than 6 carbon atoms. Examples of MCTs include: caproic acid or hexanoic acid (6 carbon atoms); caprylic acid or octanoic acid (8 carbon atoms); capric acid or decanoic acid (10 carbon atoms); and lauric acid or dodecanoic acid (12 carbon atoms). Due to their shorter chain length, MCTs are more rapidly broken down and absorbed into the body. For example, unlike longer-chain fatty acids, MCTs go straight to the liver, where they can be used as an instant energy source or turned into ketones. Ketones are substances produced when the liver breaks down large amounts of fat. In contrast with regular fatty acids, ketones can cross from the blood to the brain. This provides an alternative energy source for the brain, which ordinarily uses glucose for fuel. Because the calories contained in MCTs are more efficiently turned into energy and used by the body, they're less likely to be stored as fat.

Flavor powders may be produced in a variety of different flavor profiles, including but not limited to, natural and artificial flavors. The powder dissolves readily in warm water and is therefore useful for producing a desired flavor profile for the liquid micro-dosing product described herein. While flavor powder is preferred a variety of different flavoring agents and additives may be used consistent with the inventive concepts disclosed herein, including but not limited to, flavor liquids, extracts, concentrates, emulsions and oils for example.

In some embodiments, the cannabinoid has a bioavailability in a range between 50% and 100% and typically greater than 95% when the liquid micro-dosing product is mixed into a beverage and orally consumed by a person. Bioavailability is the proportion of the cannabinoid that enters the blood stream of a person and is able to have an active effect). The bioavailability of the cannabinoids used with the inventive concepts disclosed herein is significantly higher than bioavailability for cannabinoids traditionally infused in food and beverage products because of the improved homogenization process described herein. Homogonization provides significant non-obvious advantages over traditional methods of mixing and forming emulsions known in the art that are traditionally used to form Cannabis infused foods and beverages. Homogenization is a process used to make a mixture of two mutually non-soluble liquids the same throughout. This is achieved by turning one of the liquids into a state consisting of extremely small particles distributed uniformly throughout the other liquid.

In some embodiments, a method of producing a liquid micro-dosing product is disclosed. The method includes producing a liquid micro-dosing product that has: a distillate comprising an effective amount of a cannabinoid; water; polysorbate 80; lecithin; vegetable oil; and flavor powder. The method generally includes the following steps: mixing water and polysorbate 80 to form an aqueous solution; mixing vegetable oil, lecithin and a distillate comprising an effective amount of cannabinoid to form a distillate base solution; homogenizing the aqueous solution and the distillate base solution together to form a homogenized solution; and mixing flavor powder into the homogenized solution. An example of an embodiment of a method of producing a liquid micro-dosing product is also disclosed below in the section entitled Example.

Particle size is an important factor of gastrointestinal absorption of compounds administrated orally. The homogenization method described herein decreased cannabinoid particle size leading to faster breakdown and stabilizing of the particles when compared to non-homogenized solutions. The bioavailability of the small particle formulation is in a range of 80% to 100% (and mostly commonly 100%) compared to the 55% relative bioavailability of larger non-homogenized particles. Thus, a reduction in particle size of orally administered cannabinoid significantly increase bioavailability.

By reducing the size of the particles in the homogenized solution, it allows the particles to remain mixed for a longer period of time, increasing bioavailability, taste, shelf-life and safety and reliability of the liquid micro-dosing product. The increased bioavailability of the cannabinoid permits persons to accurately and reliably micro-dose specific amounts of cannabinoid for recreational and therapeutic purposes without fear of over-dosing, underdosing or having to perform complicated mixing procedures and measuresments in order to accurately receive the desired micro-dose of cannabinoid.

Drug particle size in oral suspension is an important factor that influences the bio-distribution and pharmaceutical properties of a compound. Particle uptake by the gastrointestinal tract is size dependent, as the uptake of smaller particles is significantly greater than larger particles. Nano- or micro-particles are also useful delivery systems to enhance oral bioavailability of many poorly absorbed drugs. Strong oral bioavailability of a drug is considered an essential component of drug development because it allows the compound to reach systemic circulation in its pure and unchanged state. For this to occur, many factors have to be considered during the development process, most importantly permeability and solubility.

In addition to homogenization, several techniques and compounds may be used or added to the to increase cannabinoid permeability, including the addition of bioavailability enhancers. Bioavailability enhancers are molecues that do not show typical drug activity but when used in combination with other compounds enhance the activity of drug molecues in several ways including increasing bioavailabity of the drug across the membrane, potentiating the drug molecule by conformational interaction acting as receptors for drug molecules and making target cells more receptive to the drug. A bioavailability enhancer is an agent capable of enhancing bioavailability and bioeffecacy of a particular drug with which it is combined, without any typical pharmacological activity of its own at the dose used. These bioavailability enhancers may also be referred to as absorption enhancers or permeability enhancers which maybe included in formulations to improve the absorption of a pharmacologically active drug. Bioavailability enhancers include, but are not limited to herbal enhancers, absorption enhancers, prodrugs, dosage form and other pharmaceutical approaches, p-glycoprotein inhibitors and various other compounds and mechanisms.

In some embodiments, a method of treating a medical condition in a subject in need of such therapy, comprising: administering to the subject a beverage containing a liquid micro-dosing product comprising a distillate comprising an effective amount of a cannabinoid; water; polysorbate 80; lecithin; vegetable oil; and flavor powder. The medical conditions to be treated, include, but are not limited to, post traumatic stress disorder, neuropathic and chronic pain, insomnia, nausea, inflammation, arthritis, migraines, cancer, crohn's disease, fibromyalgia, alzheimer's disease, multiple sclerosis, glaucoma, attention deficit hyperactivity disorder (“ADHD”), sleep apnea, appetitie loss, reducing seizures in people with epilepsy, reducing insomnia, pain relief, reducing inflammation, reducing chronic pain, reducing anxiety, relief, elation, relaxation, sedation, pain relief, energy, hunger, drowsiness, slowed perception of time or laughter for example.

In certain embodiments, the distillate containing an effective amount of cannabinoid includes an effective amount of Δ⁹-tetrahydrocannabinol, while in other embodiment the cannabinoid is Δ⁸-tetrahydrocannabinol, cannabinol or cannabidiol for example. It should be understood by one of skill in the art that the inventive concepts disclosed herein are not limited by the type of cannabinoid and that any cannabinoid or a variety or combination of cannabinoids may be used with the liquid micro-dosing product and process disclosed and claimed herein.

Distillate is a Cannabis extract in which the final product has been substantially purified of all materials and compounds except for specific cannabinoids. For example, distillate can be formed to contain pure or substantially pure Δ⁹-tetrahydrocannabinol, Δ⁸-tetrahydrocannabinol, cannabinol or cannabidiol. Distillates are Cannabis extracts that have been winterized, decarboxylated and then distilled or purified. Distillate is the base ingredient of most edibles and typically lacks any terpenes which provide a flavor, taste or aroma. One benefit of having the natural terpenes removed is being able to have complete control over the final product's taste and smell. It is a potent Cannabis oil that can be used by itself or with other Cannabis goods and products.

The process of making a Cannabis distillate generally starts with crude oil extraction, which is any process where the cannabinoids are separated from the Cannabis plant material. A crude extraction typically involves either a physical means of separation or a chemical means of separation. Physical separation techniques, such as sieving or rosin, tend to yield concentrates containing more plant impurities than chemical-based extraction methods, namely Butane Hash Oil (BHO) or supercritical fluid carbon dioxide extraction. Whether the cannabinoids are separated by physical or chemical means, the crude extract produced contains impurities that must be removed before the oil can be separated into its individual cannabinoids. The next major step in producing distillate is called winterization. It is a method to purify the crude extract of byproducts: plant waxes, fats, lipids, and chlorophyll. The crude extract is mixed with ethanol. The solution is then placed in a very cold environment for 24 to 48 hours. The impurities coagulate in the cold temperature and precipitate, or separate, falling to the bottom of the container. The crude extract and ethanol solution is then passed through a filter. After filtering, the ethanol is removed. Ethanol can be removed using a variety of techniques, such as a rotary evaporator or a falling film evaporator. The extract at this point wouldn't be very potent. THC, for example, is the well-known compound and active cannabinoid that produces an intoxicating effect. However, it's tetrahydrocannabinolic acid (THCA), that's found at this stage. THCA doesn't produce an intoxicating effect. THCA becomes THC after heat is applied. This process is called decarboxylation. THCA isn't the only cannabinoid that needs to be decarboxylated in order to interact effectively with the human body. All cannabinoids in their acid form must first be decarboxylated.

Decarboxylation is the process of removing the carboxylic acid from a cannabinoid's chemical compound. A cannabinoid is decarboxylated when it's heated to the point of eliminating the carboxylic acid. By removing that acid group, the cannabinoid can readily interact within the body and bind to the receptors in the nervous system—specifically, the cannabinoid type 1 (CB1) and cannabinoid type 2 (CB2) receptors. The point of decarboxylation depends primarily on time and temperature. For example, THCA begins to decarboxylate into THC when it's exposed to heat at 220 degrees Fahrenheit, or 104.44 degrees Celsius, or to an open flame. The final steps of making a distillate are the actual Cannabis distillation process. Using vacuum pressure and heat, individual cannabinoids and terpenes can be separated from the decarboxylated extract according to their unique boiling points and molecular weights. In a vacuum environment, where the pressure can be strictly controlled, the boiling point of can be achieved at much lower temperatures to help prevent the loss of potency.

Polysorbate 80 is a nonionic surfactant and emulsifier used to stabilize aqueous formulations. The synthetic compound is a viscous, water soluble liquid. Polysorbate 80 is derived from polyethoylated sorbitan and oleic acid. The hydrophilic groups in this compound are polyethers known as polyoxyethylene groups, which are polymers of ethylene oxide. The numeric designation following polysorbate refers to the lipophilic group, in this case, the oleic acid. The full chemical names for polysorbate 80 are: polyoxyethylene (20) sorbitan monooleate (x)-sorbitan mono-9-octadecenoate poly(oxy-1,2-ethanediyl).

Lecithin is a generic term used to describe fatty compounds occurring in animal and plant tissue which are amphiphilic, meaning that the substance attracts both water and fatty substances (and so are both hydrophilic and lipophilic). Lecithin is used for smoothing food textures, emulsifying and homogenizing liquid mixtures and repelling sticking materials. Lecithins are mixtures of glycerophospholipids including phosphatidylcholine, phosphatidylethanolamine, phosphatidylinositol, phosphatidylserine, and phosphatidic acid. Commercial lecithin, as used by food manufacturers, is a mixture of phospholipids in oil. The lecithin can be obtained by water degumming the extracted oil of seeds. It is a mixture of various phospholipids, and the composition depends on the origin of the lecithin. A major source of lecithin is soybean or sunflower oil.

Vegetable oils high in medium chain triglycerides (MCTs), including medium chain triglyceride (MCT) oil and coconut oil are edible oils high in MCTs that have been extracted from a vegetable. Triglycerides are named after their chemical structure, specifically the length of their fatty acid chains. Triglycerides consist of a glycerol molecule and there fatty acids. The majority of fat in a diet is made up of long-cain fatty acids, which contain 13-21 carbons. Short chain fatty acids typically contain fewer than 6 carbon atoms. Examples of MCTs include: caproic acid or hexanoic acid (6 carbon atoms); caprylic acid or octanoic acid (8 carbon atoms); capric acid or decanoic acid (10 carbon atoms); and lauric acid or dodecanoic acid (12 carbon atoms). Due to their shorter chain length, MCTs are more rapidly broken down and absorbed into the body. For example, unlike longer-chain fatty acids, MCTs go straight to the liver, where they can be used as an instant energy source or turned into ketones. Ketones are substances produced when the liver breaks down large amounts of fat. In contrast with regular fatty acids, ketones can cross from the blood to the brain. This provides an alternative energy source for the brain, which ordinarily uses glucose for fuel. Because the calories contained in MCTs are more efficiently turned into energy and used by the body, they're less likely to be stored as fat.

Flavor powders may be produced in a variety of different flavor profiles, including but not limited to, natural and artificial flavors. The powder dissolves readily in warm water and is therefore useful for producing a desired flavor profile for the liquid micro-dosing product described herein. While flavor powder is preferred a variety of different flavoring agents and additives may be used consistent with the inventive concepts disclosed herein, including but not limited to, flavor liquids, extracts, concentrates, emulsions and oils for example.

In some embodiments, the cannabinoid has a bioavailability in a range between 50% and 100% and typically greater than 95% when the liquid micro-dosing product is mixed into a beverage and orally consumed by a person. Bioavailability is the proportion of the cannabinoid that enters the blood stream of a person and is able to have an active effect). The bioavailability of the cannabinoids used with the inventive concepts disclosed herein is significantly higher than bioavailability for cannabinoids traditionally infused in food and beverage products because of the improved homogenization process described herein. Homogonization provides significant non-obvious advantages over traditional methods of mixing and forming emulsions known in the art that are traditionally used to form Cannabis infused foods and beverages. Homogenization is a process used to make a mixture of two mutually non-soluble liquids the same throughout. This is achieved by turning one of the liquids into a state consisting of extremely small particles distributed uniformly throughout the other liquid.

In some embodiments, a method of producing a liquid micro-dosing product is disclosed. The method includes producing a liquid micro-dosing product that has: a distillate comprising an effective amount of a cannabinoid; water; polysorbate 80; lecithin; vegetable oil; and flavor powder. The method generally includes the following steps: mixing water and polysorbate 80 to form an aqueous solution; mixing vegetable oil, lecithin and a distillate comprising an effective amount of cannabinoid to form a distillate base solution; homogenizing the aqueous solution and the distillate base solution together to form a homogenized solution; and mixing flavor powder into the homogenized solution.

Particle size is an important factor of gastrointestinal absorption of compounds administrated orally. The homogenization method described herein decreased cannabinoid particle size leading to faster breakdown and stabilizing of the particles when compared to non-homogenized solutions. The bioavailability of the small particle formulation is in a range of 80% to 100% (and mostly commonly 100%) compared to the 55% relative bioavailability of larger non-homogenized particles. Thus, a reduction in particle size of orally administered cannabinoid significantly increase bioavailability.

By reducing the size of the particles in the homogenized solution, it allows the particles to remain mixed for a longer period of time, increasing bioavailability, taste, shelf-life and safety and reliability of the liquid micro-dosing product. The increased bioavailabilty of the cannabinoid permits persons to accurately and reliably micro-dose specific amounts of cannabinoid for recreational and therapeutic purposes without fear of over-dosing, underdosing or having to perform complicated mixing procedures and measurements in order to accurately receive the desired micro-dose of cannabinoid.

Drug particle size in oral suspension is an important factor that influences the bio-distribution and pharmaceutical properties of a compound. Particle uptake by the gastrointestinal tract is size dependent, as the uptake of smaller particles is significantly greater than larger particles. Nano- or micro-particles are also useful delivery systems to enhance oral bioavailability of many poorly absorbed drugs. Strong oral bioavailability of a drug is considered an essential component of drug development because it allows the compound to reach systemic circulation in its pure and unchanged state. For this to occur, many factors have to be considered during the development process, most importantly permeability and solubility.

In addition to homogenization, several techniques and compounds may be used or added to the to increase cannabinoid permeability, including the addition of bioavailability enhancers. Bioavailability enhancers are molecues that do not show typical drug activity but when used in combination with other compounds enhance the activity of drug molecues in several ways including increasing bioavailabity of the drug across the membrane, potentiating the drug molecule by conformational interaction acting as receptors for drug molecules and making target cells more receptive to the drug. A bioavailability enhancer is an agent capable of enhancing bioavailability and bioeffecacy of a particular drug with which it is combined, without any typical pharmacological activity of its own at the dose used. These bioavailability enhancers may also be referred to as absorption enhancers or permeability enhancers which maybe included in formulations to improve the absorption of a pharmacologically active drug. Bioavailability enhancers include, but are not limited to herbal enhancers, absorption enhancers, prodrugs, dosage form and other pharmaceutical approaches, p-glycoprotein inhibitors and various other compounds and mechanisms.

As noted above, the present disclosure describes methods of administering compositions comprising an effective amount of cannabinoids for recreational use and for the treatment of certain diseases and conditions, including but not limited to, post traumatic stress disorder, neuropathic and chronic pain, insomnia, nausea, inflammation, arthritis, migraines, cancer, crohn's disease, fibromyalgia, alzheimer's disease, multiple sclerosis, glaucoma, attention deficit hyperactivity disorder (“ADHD”), sleep apnea, appetitie loss, reducing seizures in people with epilepsy, reducing insomnia, pain relief, reducing inflammation, reducing chronic pain, reducing anxiety, relief, elation, relaxation, sedation, pain relief, energy, hunger, drowsiness, slowed perception of time or laughter, in a subject.

In some embodiments, the cannabinoids may also include a pharmaceutically acceptable carrier, such as a solvent, suspending agent, or vehicle for delivering the compositions or conjugates to the subject. In addition, the cannabinoids disclosed herein may be configured to provide delayed or controlled release using formulation techniques which are well known in the art.

Practice of the methods of the present disclosure may comprise administering to a subject an effective amount of the cannabinoid in any suitable systemic and/or local formulation, in an amount effective to deliver the dosages listed herein, or other acceptable dosages as determined by the attending physician. The dosage can be administered, for example but not by way of limitation, on a one-time basis, or administered at multiple times (for example but not by way of limitation, from one to five times per day, or once or twice per week), or continuously, depending on the desired therapeutic effect.

When an effective amount of the cannabinoid is administered orally, in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol.

As noted, particular amounts and modes of administration can be determined by one skilled in the art. One skilled in the art of preparing formulations can readily select the proper form and mode of administration, depending upon the particular characteristics of the cannabinoid selected, the condition to be treated, the stage of the condition, and other relevant circumstances using formulation technology known in the art.

Several embodiments of the present disclosure, having now been generally described, will be more readily understood by reference to the following example, which is included merely for purposes of illustration of certain aspects and embodiments of the present disclosure, and is not intended to be limiting. Those skilled in the art will promptly recognize suitable and appropriate variations from the various compositions, compounds, components, procedures and methods described in the example.

### EXAMPLE

The object of this example is to describe the process for making one embodiment of a liquid micro-dosing product containing an effective amount of cannabinoid of the present disclosure.

### Formula

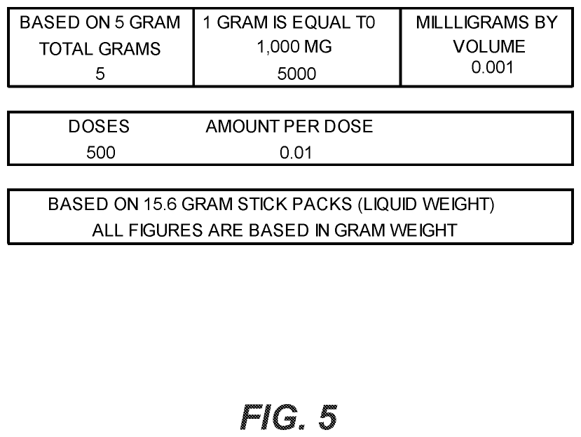

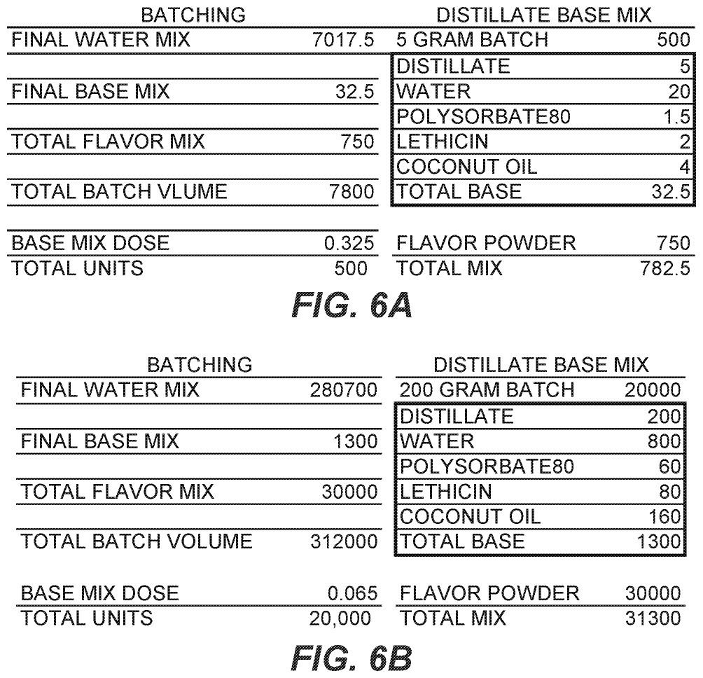

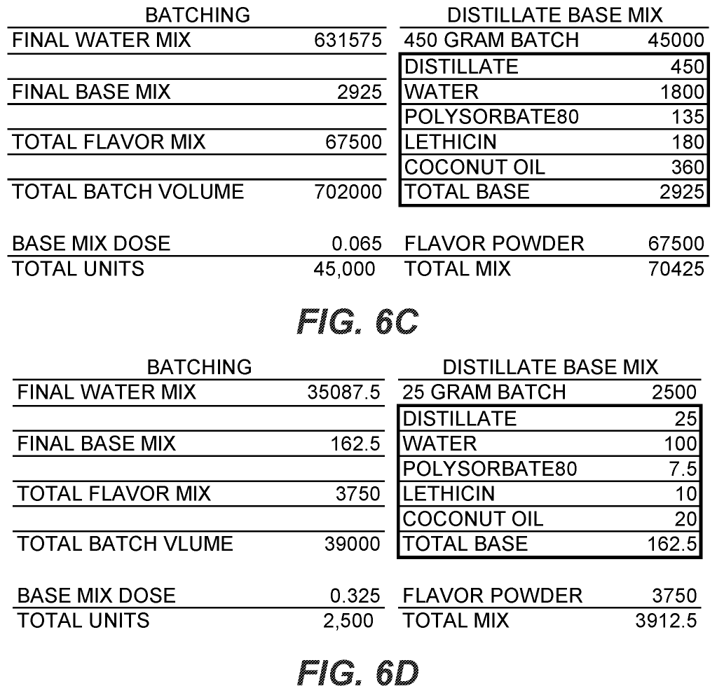

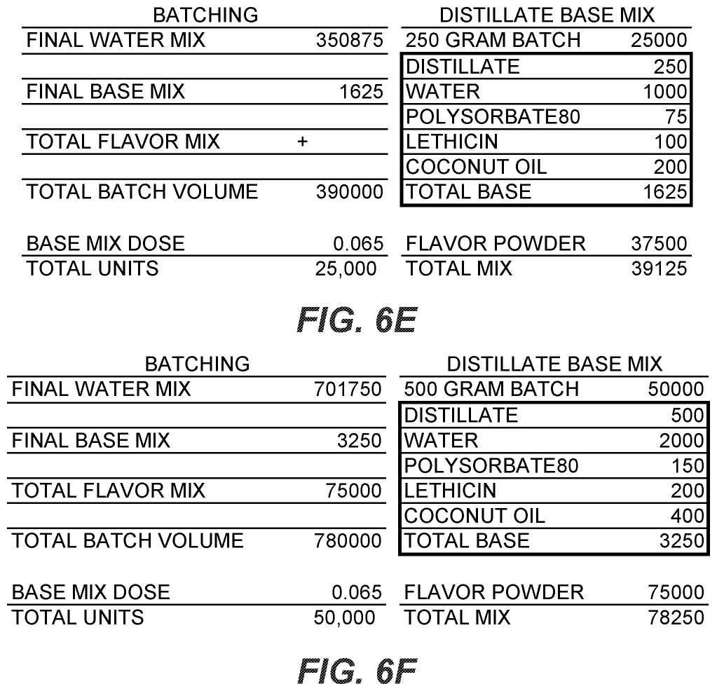

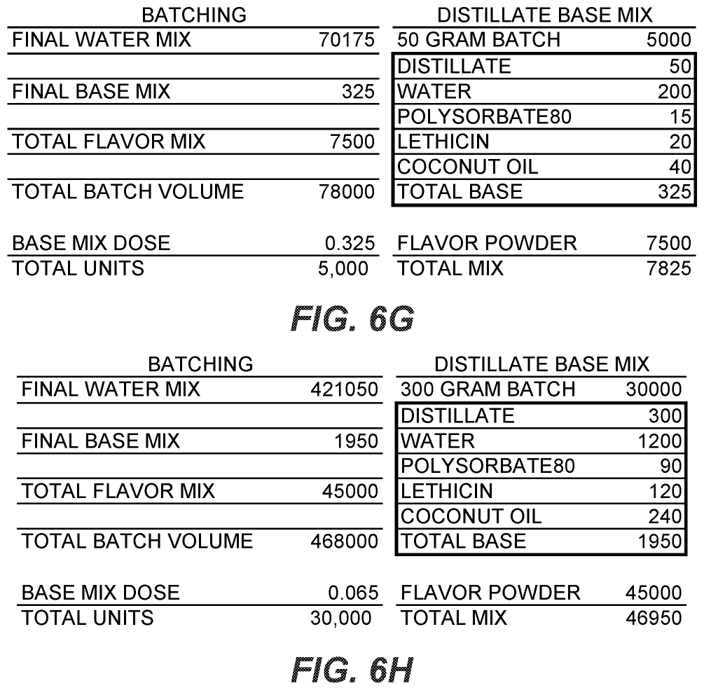

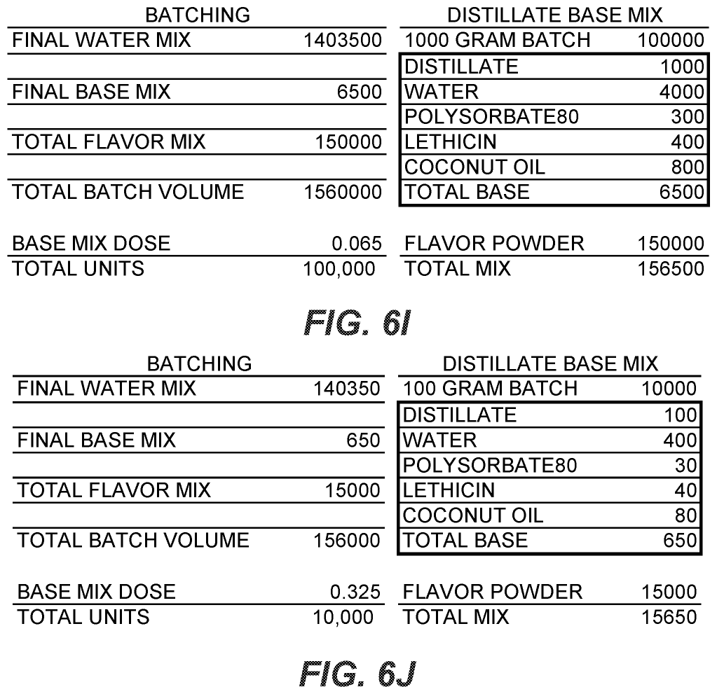

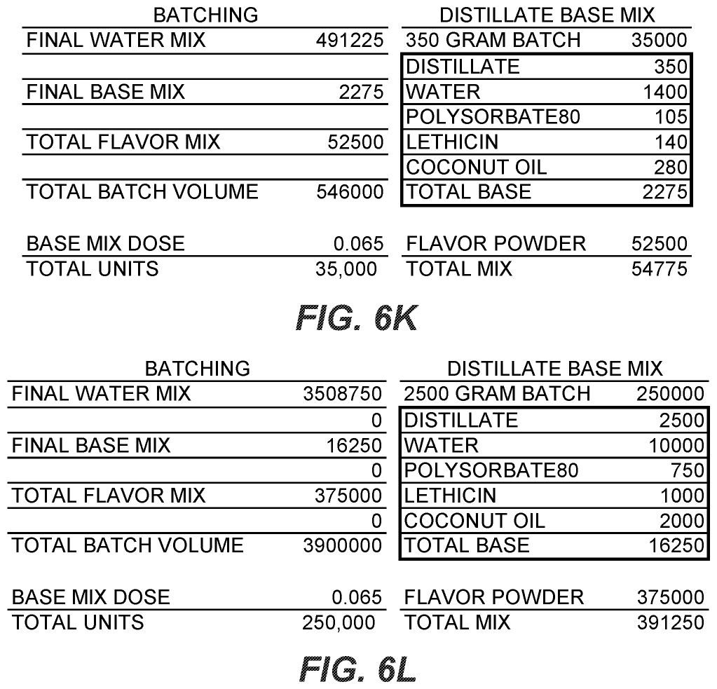

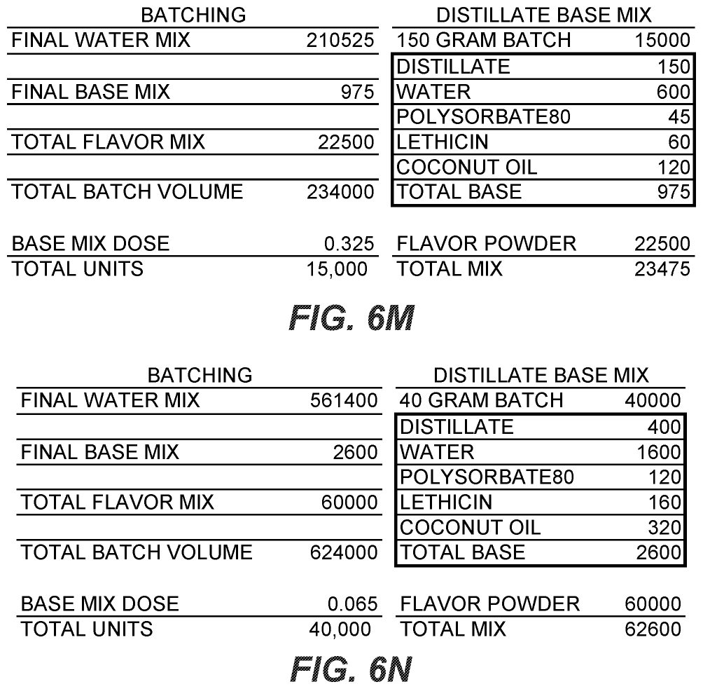

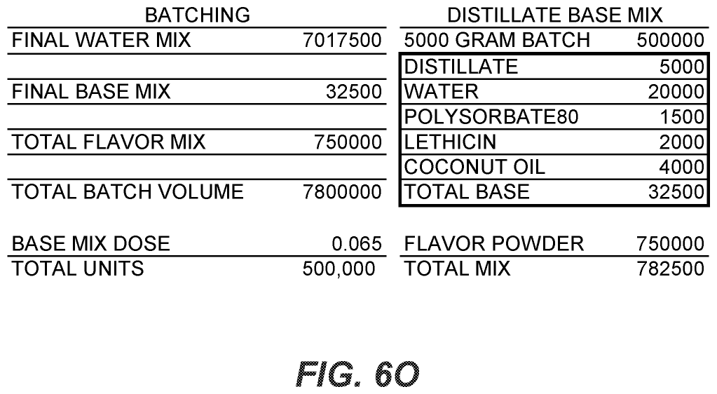

The formula for making one embodiment of a liquid micro-dosing product will vary based upon the number of desired doses to be produced in a given batch and based upon the desired strength or potency of each liquid micro-dosing product to be produced. Tables depicting the formula for a variety of different batch sizes of liquid micro-dosing product are shown in FIG. 5 and FIGS. 6A-6O.

### Methods

The example described below is based on 10 milliliters of THC distillate at 100% THC potency. If the percentage of THC potency is less than the amount of distillate used, then the base mix will need to be increased. For example, if the THC potency is 85% then the amount of distillate that would be used in the base mix will be 11.64 milliliters of distillate instead of 10 milliliters.

### Water Mix: Step 1

Add 100 milliliters of water; and

15 grams of polysorbate 80.

Fill a beaker with 100 milliliters of water. Place the beaker on a magnetic spinner. Place the magnetic spinner bar in the beaker with the water in it, start the mixer at low to medium speed. Slowly add 15 grams of polysorbate 80. Let this mix for 20 minutes or until the mixture is 100% mixed.

### Distillate Base Mix: Step 2

Add 40 grams of coconut oil, MTC oil, olive oil or any other fatty carrier oil; and

15 grams of Lecithin Soy or Sunflower; and

10 milliliters of distillate.

Preheat your distillate to no more 107 degrees Fahrenheit to make the distillate oil viscus and easy to work with. Start by pouring 40 grams of coconut oil, MTC oil, olive oil or any other fatty carrier oil into a beaker, place a magnetic spin bar into the beaker and place on a heated magnetic stirrer. Start the magnetic stirrer. Stir the carrier oil to a temperature of 101 degrees Fahrenheit. Do not let this mixture rise above 108 degrees Fahrenheit. Once the oil carrier has reached 101 degrees Fahrenheit slowly add 15 grams of Lecithin either soy or sunflower. Allow the oil carrier and the Lecithin to completely mix. This should mix for no less than 10 minutes. Once the oil and Lecithin has completely mixed then slowly add 10 milliliters of THC distillate. Allow this to mix thoroughly on the heated magnetic stirrer, but not less than 15 minutes.

### Homogenization: Part 1: Step 3

Place the beaker of water and polysorbate 80 in the harmonic, ultra-sonic homogenizer. Start homogenizing the water and polysorbate 80 at 50% power and at a frequency of 20 MHz-24.95 MHz. Allow this to homogenize for 45 seconds. After homogenizing for 45 seconds, increase the power to 85% (the frequency does not change) then start slowly introducing the distillate base mix. This will be done with a syringe straight into the mix while it is homogenizing. Once the distillate base mix has been completely introduced to the water mix then the power must increase to 100% for 1 minute and 30 seconds. Do not let the mix temperature rise above 108 degrees Fahrenheit.

After 1 minute and 30 seconds remove the entire mixture for the homogenizer. Place the beaker into and ice bath. Allow the mixture to cool for 10 minutes. After cooling for 10 minutes place the beaker back into the homogenizer while still in the ice bath and start the homogenization process at 100% power for 3 minutes.

### Homogenization: Part 2: Step 4

Once the water and distillate base mix is completely homogenized remove the beaker from the homogenizer. Place the beaker on a magnetic spinner and add 20 milliliters of water. While the mixture is mixing on the magnetic stirrer, slowly pour 50 grams of concentrated flavor powder. Allow this mixture to fully mix and the powder to fully dissolve into the mixture. Once the mixture is completely mixed place the beaker back into an ice bath and place back into the homogenizer. Start the homogenizer at 100% at the same frequency as above. Allow this mix to homogenize for 3 minutes. Stop the homogenizer let cool for 3 minutes then start the homogenizer at 100% for 2 more minutes. At this point the homogenization process is complete. At this point you have 1,000 doses of THC. This dose will be 0.16 ml/g per serving.

### Final Steps

Pour into a container 14,300 milligrams of distilled water, next stir into the water slowly 1,050 grams of concentrated flavor mix. Allow this to mix for 10 minutes on a magnetic mixer. Once this has mixed for 10 minutes add the base mix 250 grams, consisting of the water base mix, flavoring and the distillate base mix. Allow this to thoroughly mix for 20 minutes on a magnetic stirrer. This example will make 1,000 doses of liquid micro-dosing products (also referred to herein as beverage enhancers). Each complete packet of liquid micro-dosing product will contain exactly 10 milligrams of THC and an over all net weight of 15.6 grams per liquid serving.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. A liquid micro-dosing product comprising: a distillate comprising an effective amount of a cannabinoid; water; polysorbate 80; lecithin; vegetable oil; and flavor powder.

2. The liquid micro-dosing product of claim 1 further comprising a bioavailability enhancer.

3. The liquid micro-dosing product of claim 1, wherein the cannabinoid is Δ⁹-tetrahydrocannabinol.

4. The liquid micro-dosing product of claim 1, wherein the cannabinoid is Δ⁸-tetrahydrocannabinol.

5. The liquid micro-dosing product of claim 1, wherein the cannabinoid is cannabinol.

6. The liquid micro-dosing product of claim 1, wherein the cannabinoid is cannabidiol.

7. The liquid micro-dosing product of claim 1, wherein the vegetable oil is coconut oil.

8. The liquid micro-dosing product of claim 1, wherein the cannabinoid has a bioavailability greater than 95% when the liquid micro-dosing product is mixed into a beverage and orally consumed by a person.

9. The liquid micro-dosing product of claim 1, wherein each of the water; polysorbate 80; lecithin; vegetable oil; and flavor powder are present in the liquid micro-dosing product at about 0.260% to 0.280%.

10. The liquid micro-dosing product of claim 1, wherein the distillate comprising an effective amount of cannabinoid is present in the liquid micro-dosing product at about 0.05% to 0.20%.

11. A method of producing a liquid micro-dosing product comprising: a distillate comprising an effective amount of a cannabinoid; water; polysorbate 80; lecithin; vegetable oil; and flavor powder, the method comprising the following steps:
mixing water and polysorbate 80 to form an aqueous solution;
mixing vegetable oil, lecithin and a distillate comprising an effective amount of cannabinoid to form a distillate base solution;
homogenizing the aqueous solution and the distillate base solution together to form a homogenized solution; and
mixing flavor powder into the homogenized solution.

12. A method of treating a medical condition in a subject in need of such therapy, comprising:
administering to the subject a beverage containing a liquid micro-dosing product comprising a distillate comprising an effective amount of a cannabinoid; water; polysorbate 80; lecithin; vegetable oil; and flavor powder.

13. The method of claim 12, wherein the cannabinoid is Δ⁹-tetrahydrocannabinol.

14. The method of claim 12, wherein the cannabinoid is Δ⁸-tetrahydrocannabinol.

15. The method of claim 12, wherein the cannabinoid is cannabinol.

16. The method of claim 12, wherein the cannabinoid is cannabidiol.

17. The method of claim 12, wherein the vegetable oil is coconut oil.

18. The method of claim 12, wherein the cannabinoid has a bioavailability greater than 95% when the liquid micro-dosing product is mixed into a beverage and orally consumed by a person.

19. The method of claim 12, wherein each of the water; polysorbate 80; lecithin; vegetable oil; and flavor powder are present in the liquid micro-dosing product at about 0.260% to 0.280%.

20. The method of claim 12, wherein the distillate comprising an effective amount of cannabinoid is present in the liquid micro-dosing product at about 0.05% to 0.20%.
